(19) United States
(12) Patent Application Publication
(10) Pub. No.: US 2009/0065005 A1
(43) Pub. Date: Mar. 12, 2009

(54) Nose mask assembly to be worn during sleep having a suspension support bracket and a retractable line

(76) Inventor: Abraham J. Ades, Brooklyn, NY (US)

Correspondence Address: Ezra Sutton, Esq., Suite 201, 900 Route 9, Woodbridge, NJ, 07095, US

(21) Appl. No.: 11/899,865

(22) Filed: Sep. 6, 2007

(51) Int. Cl. A61M 16/06

(52) U.S. Cl. 128/205.25; 128/207.13

(57) ABSTRACT

A nose mask assembly to be worn during sleep including a nose mask, an air hose, a suspension support bracket connected to the air hose, a retractable line connected to the air hose, and a ball joint or a flexible silicone rubber tubing member connected to the nose mask for supplying continuous positive air pressure to a user. The nose mask includes an intake port for an air hose, an opening for an exhalation air valve therein, an air chamber for receiving air, and a contoured nose section for fitting on the nose of the user. The nose mask includes holding means in the form of an expandable headband for retaining the nose mask on the head of the user. The intake port includes the ball joint swivel member or a flexible silicone rubber tubing member for swiveling of the air hose relative to the nose section of the nose mask allowing the user to move side to side without having the air hose pulling on the nose mask during sleep movements of the user. The suspension support bracket and the retractable spring-loaded line mechanism are for suspending the air hose over the user's head while sleeping for relieving the nose mask of any pulling action being applied by the suspended air hose during sleep movements of the wearer. The suspension support bracket is connected to a support base plate or a support base bar being located under a CPAP machine which acts as a stabilizing counterweight to the suspension support bracket for suspending the air hose over the head of the user.

### FIELD OF THE INVENTION

The present invention relates to a sleeping nose mask assembly that provides for a lightweight, comfortable and customized molded nose mask and a suspension support bracket that cannot be pulled, twisted or moved out of place by head movements of the wearer while sleeping. More particularly, the sleeping nose mask assembly includes a nose mask, an air hose, a suspension bracket and a retractable spring-loaded line mechanism connected to a clamp for suspending the air hose over the wearer's head in order to relieve any tension on the suspended air hose and the mask during sleep movements of the wearer.

### BACKGROUND OF THE INVENTION

It is well known in the art that many people use nose masks for breathing problems that often result in sleep disorder breathing by the wearer having difficulty sleeping, snoring, or other more serious conditions such as obstructure sleep apnea. As technology advances, people with such breathing problems demand increased performance and comfort. Previous devices for improving a user's breathing have included custom fitted masks that help deliver air or another suitable gas to the user's nose at positive pressure to help force open the user's breathing. However, previous masks may be relatively expensive due to the expertise often required to design, manufacture, and fit the masks to each particular user.

Furthermore, previous masks have often been fitted to the user's unique bone structure and facial features rather poorly, if at all. As a result, previous masks have often failed to adequately prevent leakage from around the perimeter of the mask, have caused extreme discomfort for users, and have been relatively ineffective in treating breathing problems such as sleep disorder breathing. Also, previous nose masks are not comfortable to the wearer during sleep as the air hose does not allow the wearer to move side to side without the air hose pulling on the nose mask during sleep movements of the wearer.

There remains a need for a suspension bracket connected to the air hose and a retractable spring-loaded line mechanism thereon for suspending the air hose over the wearer's head while sleeping in order to relieve the nose mask from applying any pulling pressure by the suspended air hose during sleep movements of the wearer. Additionally, there remains a need for the nose mask having a ball joint swivel member or a flexible silicone ribbed tubing member relative to the nose section of the nose mask for allowing the wearer's head to move side to side without having the suspended air hose applying pulling or pressure on the nose mask during sleep movements of the wearer.

### DESCRIPTION OF THE PRIOR ART

Nose masks, custom fitted masks, respiratory facial masks, nasal masks, gas delivery masks, gas delivery masks and the like having various designs, configurations, structures and materials of construction have been disclosed in the prior art. For example, U.S. Patent Application Publication No. 2006/0283457 to WOODARD et al. discloses a system for adjusting a gas delivery mask. The system includes a first housing portion, a first rim flexibly coupled to the first housing portion by a flexible member, a second housing portion coupled to the first housing portion, a second rim associated with the second housing portion, and a ball rotatably disposed between the first and second rims and having a gas passageway extending therethrough. The ball joint may be maintained in contact with both the first and second rims by a resiliency of the flexible member. This prior art publication does not disclose or teach the specific structure of a sleeping nose mask assembly having a suspension support bracket for user wear during sleep apnea.

U.S. Pat. No. 6,857,428 to THORNTON discloses a method of forming a custom fitted nose mask including transitioning a mask blank including a thin sheet of deformable material from a non-deformable state into a deformable state. The mask blank is applied against at least a portion of a user's face surrounding the user's nostrils while the mask blank is in the deformable state. The mask blank is deformed against the portion of the user's face to cause the mask blank to conform substantially optimally to the user's unique bone structure and facial features. The mask blank once conformed substantially optimally to the user's unique bone structure and facial features becoming the custom fitted mask. The custom fitted nose mask is allowed to transition from the deformable state into the non-deformable for use. This prior art patent does not disclose or teach the specific structure of a sleeping nose mask assembly having a suspension support bracket for user wear during sleep apnea.

U.S. Pat. No. 5,918,598 to BELFER et al. discloses a strapless respiratory facial mask for attachment to the wearer's face. This device includes a mask having a central section and three edges forming a generally triangular configuration for covering and surrounding the nose of the wearer; wherein the first and second edges of the mask extend along the opposite sides of the nose wearer, and the third edge extends across the upper lip area of the wearer. The central section of the mask is formed of a thermoplastic material and has a first central opening formed therein for making an external connection to the mask, and having a second central opening below the first central opening and an external tubular section connected to the second central opening for making a connection to a gas supply. The mask further includes an elastic and moldable cushioning material connected to the first, second and third edges of the mask to form a peripheral sealing section for engaging the facial contours and skin of the wearer's face. The peripheral sealing section includes first, second and third sealing section edges. In addition, the mask also includes a plurality of sealing strips formed of the cushioning material for attachment to one or more of the first, second or third sealing section edges for providing an increased sealing area for the mask. This prior art patent does not disclose or teach the specific structure of a sleeping nose mask assembly having a suspension support bracket for user wear during sleep apnea.

None of the aforementioned prior art publications and patents teach or disclose a molded and contoured sleeping nose mask assembly that includes a nose mask having a ball joint swivel or a flexible silicone ribbed tubing member and a suspension support bracket attached to an air hose over the wearer's head in order to relieve any tension applied by the suspended air hose during sleep movements of the wearer.

Accordingly, it is an object of the present invention to provide a sleeping nose mask assembly that is lightweight, comfortable and custom molded to the wearer's face, as well as having a suspension support bracket and retractable line that cannot be pushed, pulled, twisted or moved out of place by side to side head movements of the wearer while sleeping.

Another object of the present invention is to provide a sleeping nose mask assembly having a suspension support bracket and a retractable spring-loaded line mechanism for suspending the air hose over the wearer's head in order to relieve any tension applied by the suspended air hose during sleep movements of the wearer.

Another object of the present invention is to provide a sleeping nose mask assembly having a suspension support bracket and an intake port on the sleeping nose mask for receiving a detachable ball joint swivel or a flexible silicone ribbed tubing member therein for swiveling of the attached air hose relative to the nose section of the nose mask allowing the wearer to move side to side without having the suspended air hose pulling or pushing on the nose mask during sleeping movements of the wearer.

Another object of the present invention is to provide a sleeping nose mask assembly having a suspension support bracket connected to a support base plate under a CPAP machine which acts as a stabilizing counterweight to the suspension support bracket for suspending the air hose over the head of the wearer.

Another object of the present invention is to provide a sleeping nose mask assembly and a suspension support bracket that are easy to use, easy to clean for repeated use, and durable for extended wearer use.

Another object of the present invention is to provide a sleeping nose mask assembly having a suspension support bracket being made from flexible metal tubing, semi-rigid plastic tubing or flexible plastic I-bar, or a flat metal bar.

A further object of the present invention is to provide a sleeping nose mask assembly having a suspension support bracket that can be mass-produced in an automated and economical manner and is readily affordable by the user to reduce sleep apnea.

### SUMMARY OF THE INVENTION

In accordance with the present invention, there is provided a nose mask assembly to be used during sleep including a nose mask, an air hose, a suspension support bracket connected to the air hose, a retractable line connected to the air hose, and a ball joint or a flexible silicone ribbed tubing member connected to the nose mask for supplying continuous positive air pressure to a user. The nose mask includes an intake port for an air hose, an opening for an exhalation air valve therein, an air chamber for receiving air, and a contoured nose section for fitting on the nose of the user. The nose mask includes holding means in the form of an expandable headband for retaining the nose mask on the head of the user. The intake port includes the ball joint swivel member or a flexible silicone ribbed tubing member for swiveling of the air hose relative to the nose section of the nose mask allowing the user to move side to side without having the air hose pulling on the nose mask during sleep movements of the user. The suspension support bracket and the retractable spring-loaded line mechanism are for suspending the air hose over the user's head while sleeping for relieving the nose mask of any pulling action being applied by the suspended air hose during sleep movements of the wearer. The suspension support bracket is connected to a support base plate or a support base bar being located under a CPAP machine which acts as a stabilizing counterweight to the suspension support bracket for suspending the air hose over the head of the user.

### BRIEF DESCRIPTION OF DRAWINGS

FIG. 1 is a perspective view of the sleeping nose mask assembly having a suspension support bracket of the preferred embodiment of the present invention showing all of the major component parts thereof;

FIG. 2 is a front perspective view of the sleeping nose mask assembly of the present invention showing a nose mask in an assembled state and in an operational mode thereto;

FIG. 3 is a rear perspective view of the sleeping nose mask assembly of the present invention showing the nose mask having a contoured nose section, an air chamber and a head strap;

FIG. 4 is a top perspective view of the sleeping nose mask assembly of the present invention showing the nose mask having a mask housing with a ball joint swivel member and an air hose;

FIG. 5 is a front exploded perspective view of the sleeping nose mask assembly of the present invention showing the nose mask housing, the ball point swivel member and the air hose;

FIG. 6 is a side perspective view of the sleeping nose mask assembly of the present invention showing the contoured nose section, an intake port receiving the ball joint swivel member and the air hose thereon;

FIG. 7 is a perspective view of the suspension support bracket of the present invention showing a suspension support bracket, a retractable spring-loaded line mechanism having a spring-activated pulley with a nylon line thereon and the air hose;

FIG. 8 is a perspective view of the suspension support bracket of the present invention showing the suspension support bracket, the air hose, a support base plate and a CPAP machine;

FIG. 9 is a side perspective view of the sleeping nose mask assembly of the present invention showing the contoured nose section, the intake port receiving a flexible silicone ribbed tubing member and the air hose thereon; and

FIG. 10 is a perspective view of the suspension support bracket of the present invention showing the suspension support bracket in the form of a flat metal bar, a support flat metal bar member, the air hose and the CPAP machine.

### DETAILED DESCRIPTION OF THE PREFERRED EMBODIMENT 10

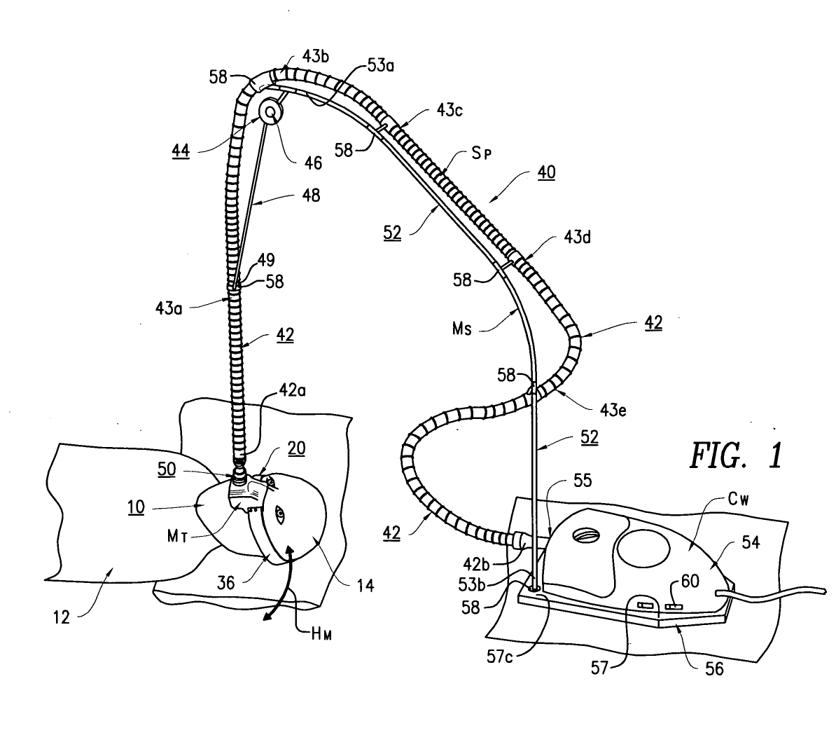

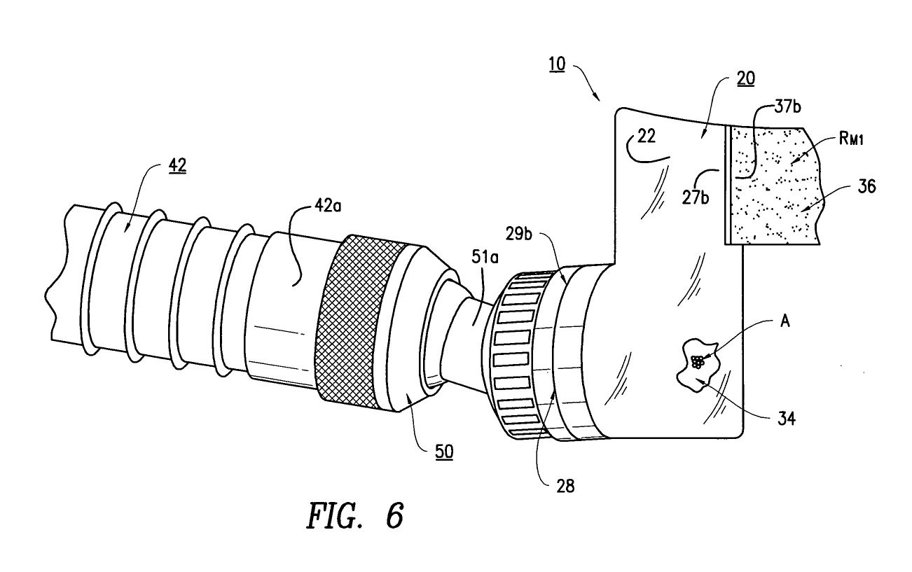

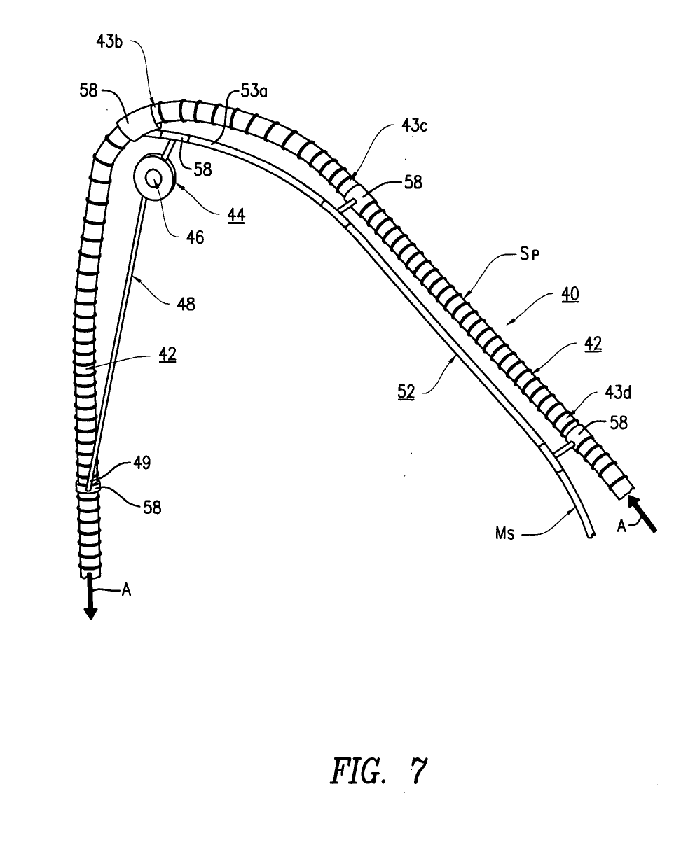

The sleeping nose mask assembly 10 having a suspension bracket 52 of the preferred embodiment of the present invention is represented in detail by FIG. 1 through 10 of the patent drawings. The sleeping nose mask assembly 10 having a suspension support bracket 52 thereon is used for suspending an air hose 42 over a wearer's head 14 in order to reduce any head movement H_{M} during sleep (for user's having sleep apnea), as depicted in FIG. 1. This allows the suspension support bracket 52 relative to the nose mask 10 from any pushing, pulling, twisting or moving out of place by side to side head movements H_{M} of the wearer 12.

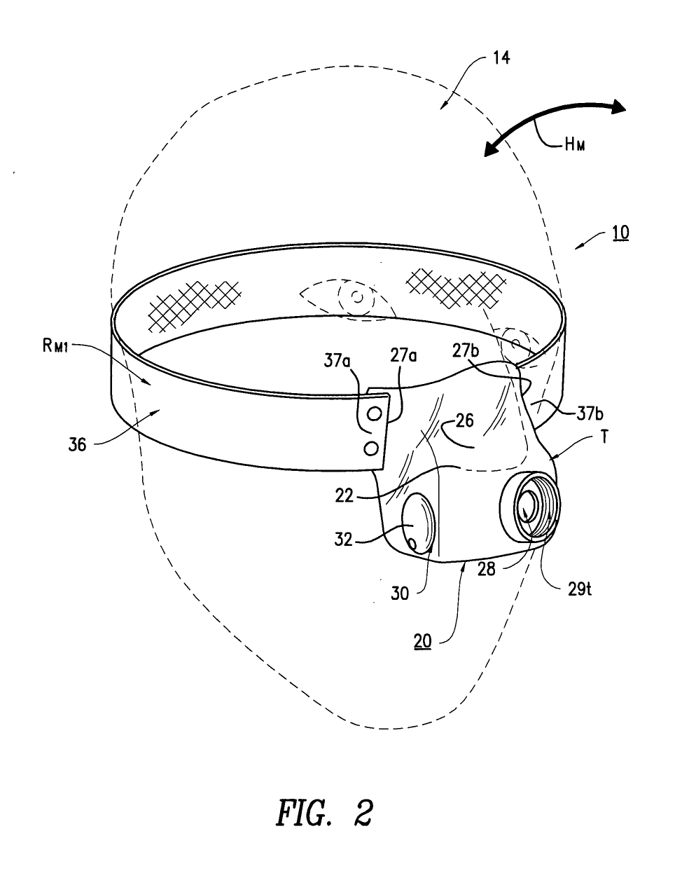

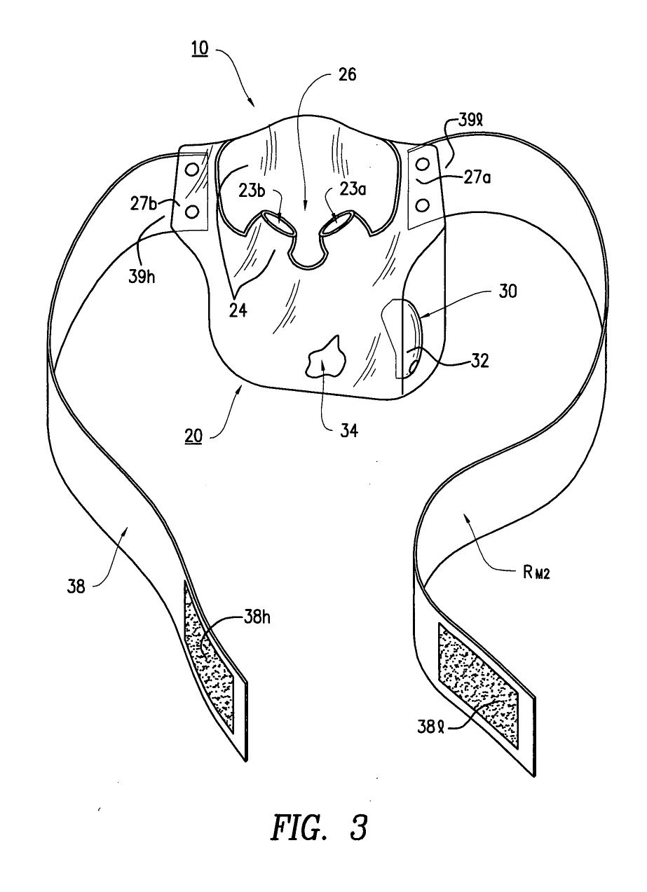

As shown in FIGS. 1 through 7 of the drawings, the sleeping nose mask assembly 10 having the suspension support bracket 52 thereon includes a mask housing 20 being substantially triangularly-shaped T. The mask housing 20 includes an exterior wall surface 22, an interior wall surface 24, and a contoured nose section 26 for sitting on and over a nose 16 area of the wearer 12. The mask housing 20 also includes an intake port 28 for an air hose 42, an opening 30 for an exhalation air valve 32, and an air chamber 34 contoured for receiving air A from the air hose 42. The nose mask 10 further includes retaining means RM1 and RM2 for retaining and holding the nose mask 10 on the head 14 of the wearer 12. Retaining means RM1 can be in the form of an expandable headband 36 having opposing ends 37a and 37b, wherein each of the opposing ends 37a and 37b are attached to opposing sides 27a and 27b, respectively, of the contoured nose section 26. Another retaining means RM2 can be in the form of an adjustable headband 38 having hook and loop straps 38 h and 38 l each having opposing proximal ends 39 h and 39 l, respectively, wherein each of the opposing proximal ends 39h and 39 l are attached to opposing sides 27a and 27b, respectively, of the contoured nose section 26, as shown in FIGS. 2 and 3 of the drawings. The interior wall surface 22 of the contoured nose section 26 includes a pair of spaced apart interior nasal openings 23a and 23b for allowing air or oxygen to be received through nasal openings 23a and 23b for the wearer's nose 16. The nose mask 10 is made from moldable thermoplastic materials M_{T}. It is understood that the intake port 28 can include a threaded opening 29t or a bayonet-type opening 29b or other connecting means for attaching the ball joint swivel member 50 therein.

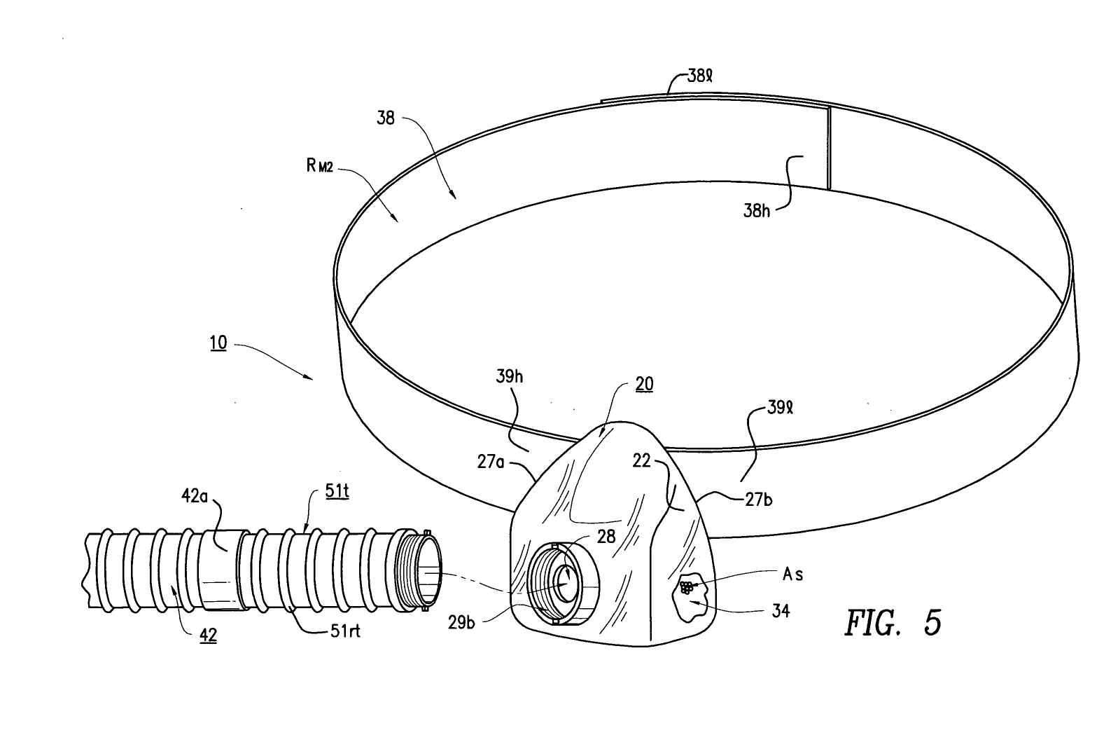

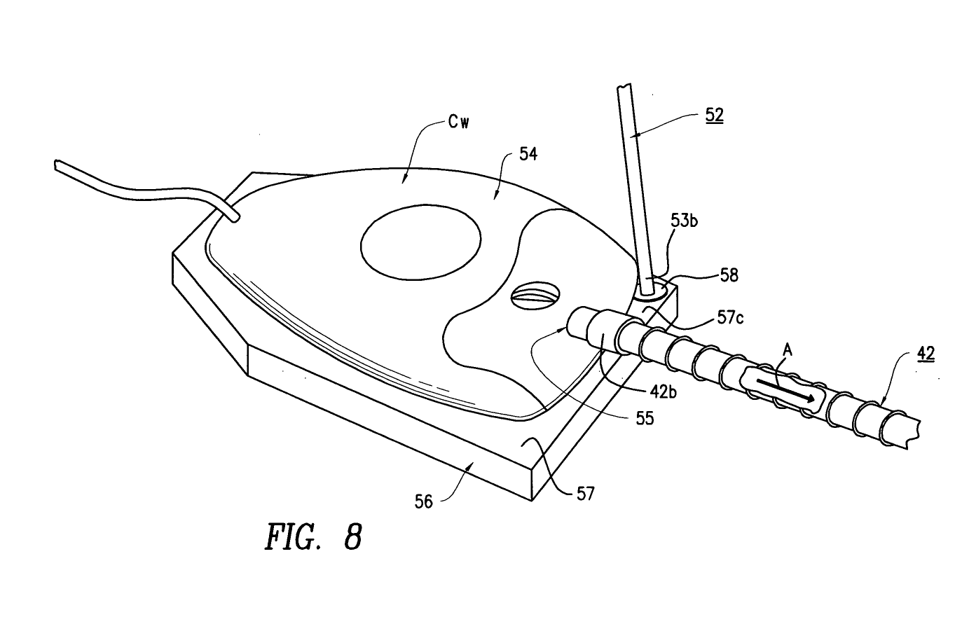

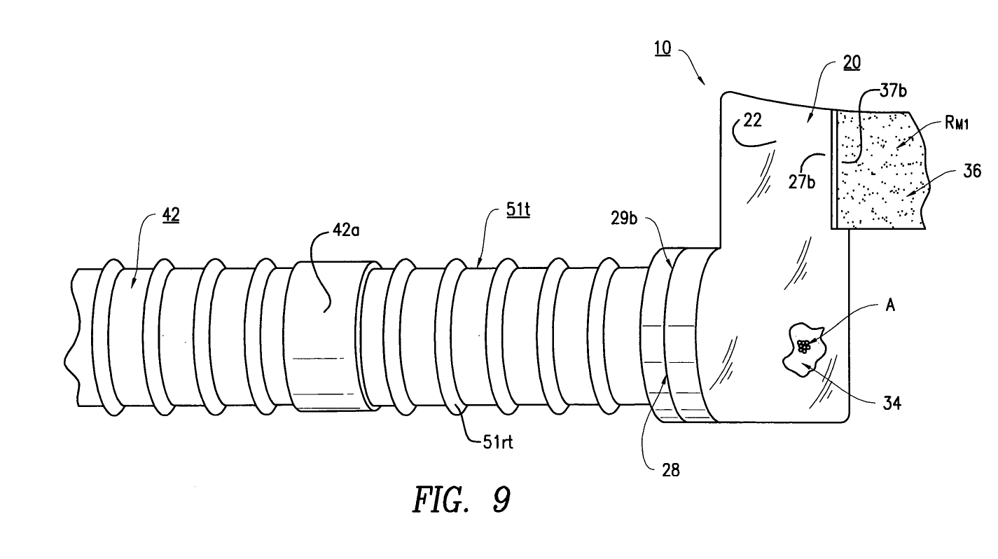

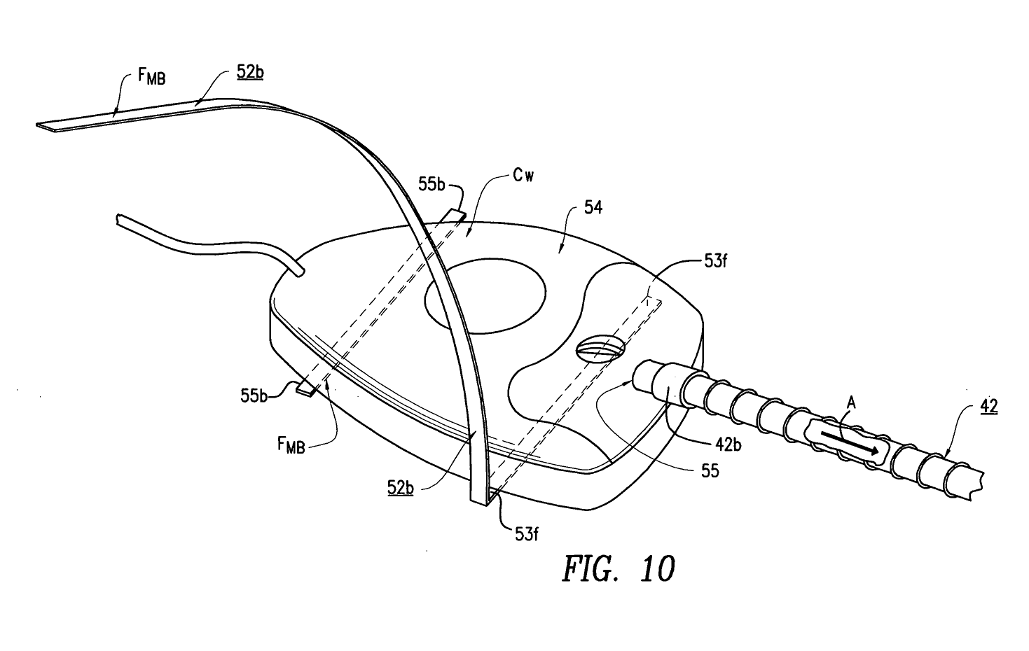

The suspension support bracket 52, as shown in FIGS. 1, 6, 7 and 8, is detachably connected to an air hose 42 and a retractable spring-loaded line mechanism 44 having a spring-activated pulley 46 with a nylon line 48 thereon, and a ball joint swivel member 50. In an alternate design, as shown in FIG. 9, the ball joint swivel member 50 is replaced with a flexible ribbed tubing member 51t having ribbed tubing 51rt being capable of moving and swiveling of the air hose 42 (See FIGS. 5 and 9). The suspension support bracket 52 is connected to a continuous air pressure pump (CPAP) machine 54 and a support base plate 56 positioned under the CPAP machine 54 which acts as a stabilizing counterweight CW to the suspension support bracket 52 for suspending the air hose 42 over the head 14 of the user 12. Air hose 42 includes a first connecting end 42a and a second connecting end 42b. The air hose 42 is used for supplying continuous positive air pressure A to the user 12 using the CPAP machine 54. In an alternate design embodiment, as shown in FIG. 10, the suspension support bracket 52b is in the form of a flat metal bar F_{MB}. A bottom section 53f is positioned under the CPAP machine 54 and has replaced the support base plate 56 (see FIG. 8). Further, another flat metal bar being a support base bar 55b is positioned under the CPAP machine 54 to prevent the CPAP machine 54 from rocking or moving when in operation.

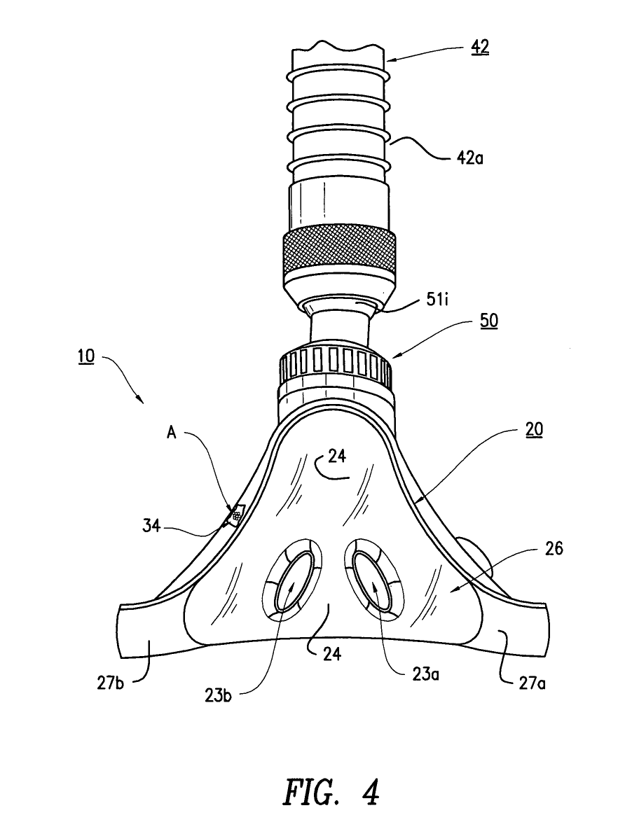

As shown in FIGS. 4, 5 and 6, the intake port 28 of mask housing 20 is for receiving the ball joint swivel member 50 and the first connecting end 42 of air hose 42, respectively, thereto. This enables the ball joint swivel member 50 to swivel the air hose 42 relative to the contoured nose section 26 of mask housing 20 in order for the wearer 12 to move side to side H_{M} without having the air hose 42 pulling or pushing on the nose mask 10 during sleep movements of the user 12.

The suspension support bracket 52 includes a first end 53a and a second end 53b. The first end 53a of suspension support bracket 52 is attached to the spring activated pulley 46 of the line mechanism 44 using a detachable attaching clamping member 58 thereon. The second end 53b of suspension support bracket 52 is attached to an upper surface 57 of support base plate 56 by the detachable attaching clamping member 58 thereto. The support base plate 56 is made from materials selected from the group consisting of metals, plastics or woods being used also as a weighted plate to the CPAP machine 54 which sits on the support base plate 56, as shown in FIG. 1 of the drawings. The suspension support bracket 52 is also detachably connected to the air hose 42 at various hose connecting points 43a, 43b, 43c, 43d and 43e, by the plurality of detachable attaching clamping members 58 in order to suspend the air hose 42 above the head 14 of the wearer 12, as depicted in FIG. 1 of the drawings. The suspension support bracket 52 has a length in the range of 36 inches to 48 inches±3 inches. The suspension support bracket 52 is made from materials Ms selected from the group consisting of flexible aluminum tubing, flexible stainless steel tubing, semi-rigid plastic tubing and semi-rigid plastic I-bar or a flat metal bar (see FIG. 10).

Additionally, the nylon line 48 on spring-activated pulley 46 has a length of at least 20 inches. The nylon line 48 includes a distal end 49 which attaches to the hose section connecting point 43a, as shown in FIG. 1, by one of the detachable attaching clamping members 58 on air hose 42, wherein the connecting point 43a is in close proximity to the nose mask 10, as depicted in FIG. 1 of the patent drawings.

### Operation of the Present Invention

As shown in FIGS. 1, 2, 6 and 7 of the drawings, the sleeping nose mask assembly 10 and the suspension support bracket 52 are assembled and are operated in the following manner: The user 12 initially starts the assembly process by attaching the suspension support bracket 52 to the air hose 42 at hose connecting points 43b, 43c, 43d and 43e using the detachable attaching clamping members 58, as depicted in FIG. 1. The user 12 then attaches first end 53a of suspension support bracket 52 to the spring-activated pulley 46 of line mechanism 44 using another detachable attaching clamping member 58 at connecting point 43b. Then the user 12 attaches the second end 53b of suspension support bracket 52 to a corner section 57c of the upper surface 57 of support base plate 56 by the detachable attaching clamping member 58. The user 12 now places the CPAP machine 54 on the upper surface 57 of the support base 56 which acts as a stabilizing counter-weight CW for keeping the suspension support bracket 52 in a suspended position Sp with the air hose 42 when fully assembled, as shown in FIG. 1. The next step has the user 12 connecting the distal end 49 of nylon line 48 on the spring-activated pulley 46 to the hose section connecting point 43a by attaching the clamping member 58 to the distal end 49 of nylon line 48 and to air hose 42. The connecting point 43a is in close proximity to nose mask 10, as depicted in FIG. 1.

The user now detachably connects the first connecting end of air hose 42 to an intake manifold 511 on the ball joint swivel member 50 and the user now connects a threaded male element 51m on ball joint swivel member 50 to the threaded opening 29t of intake port 28 on mask housing 22. The last step has the user 12 connecting the second connecting end 42b of air hose 42 to an inlet opening 55 on the CPAP machine 54. The assembled sleep nose mask assembly 10 is in an operational mode wherein the user 12 puts on the expandable headband 36 or the adjustable headband 38 with hook and loop straps 38h and 38l on his or her head 14 and places the contoured nose section 26 over the nose area 16 of user 12. The user then switches the CPAP machine 54 to an “ON” position, using switch 60, where then pressurized air A is sent through air hose 42, ball joint swivel member 50, intake port 28, and air chamber 34, respectively, such that the user 12 breathes the air A through interior nasal openings 23a and 23b of contoured nose section 26 for inducing sleep, as depicted in FIGS. 1 and 8. As the user 12 breathes air A, the air A is expelled from the sleeping nose mask housing 20 through the exhalation air valve 32, as shown in FIG. 2 of the drawings, such that the sleeping nose mask assembly 10 is now in a fully operational mode.

In the fully operational mode, the sleeping nose mask assembly 10 and the suspension support bracket 52, as shown in FIG. 1, cannot be pushed, pulled, twisted or moved out of place by side to side head movements H_{M} of the wearer 12 while sleeping. Further, the suspension support bracket 52 relieves any tension on the suspended air hose 42 and the nose mask 20 while the air hose 42 is in the suspended position Sp is over the wearer's head 14 during sleep movements of the wearer 12.

### ADVANTAGES OF THE PRESENT INVENTION

Accordingly, an advantage of the present invention is that it provides for a sleeping nose mask assembly that is lightweight, comfortable and custom molded to the wearer's face, as well as, having a suspension support bracket that cannot be pushed, pulled, twisted or moved out of place by side to side head movements of the wearer while sleeping.

Another advantage of the present invention is that it provides for a sleeping nose mask assembly having a suspension support bracket and a retractable spring-loaded line mechanism for suspending the air hose in order to relieve any tension applied by the suspended air hose during sleep movements of the wearer.

Another advantage of the present invention is that it provides for a sleeping nose mask assembly having a suspension support bracket and an intake port on the sleeping nose mask for receiving a detachable ball joint swivel member therein for swiveling of the attached air hose relative to the nose section of the nose mask or a flexible silicone ribbed tubing member allowing the wearer to move side to side without having the suspended air hose pulling or pushing on the nose mask during sleeping movements of the wearer.

Another advantage of the present invention is that it provides for a sleeping nose mask assembly having a suspension support bracket connected to a support base plate under a CPAP machine which acts as a stabilizing counterweight to the suspension support bracket for suspending the air hose over the head of the wearer.

Another advantage of the present invention is that it provides for a sleeping nose mask assembly having a suspension support bracket that are easy to use, easy to clean for repeated use, and durable for extended wearer use.

Another advantage of the present invention is that it provides for a sleeping nose mask assembly having a suspension support bracket being made from flexible metal tubing, semi-rigid plastic tubing or flexible metal tubing or flexible plastic I-bar or a flat metal bar.

A further advantage of the present invention is that it provides for a sleeping nose mask assembly having a suspension support bracket thereon that can be mass-provided in an automated and economical manner and is readily affordable by the user to reduce sleep apnea.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1. A nose mask assembly to be used during sleep including a nose mask, an air hose, a suspension support bracket connected to the air hose, a retractable line connected to the air hose, and a flexible member connected to the nose mask for supplying continuous positive air pressure to a user, comprising:
a) a nose mask including an intake port for an air hose, an opening for an exhalation air valve, an air chamber contoured for receiving air, and a contoured nose section for fitting on the nose of the user;
b) said nose mask including holding means for retaining said nose mask on the head of the user;
c) said intake port including a flexible member for swiveling of said air hose relative to the nose section of said nose mask for the user to move side to side without having said air hose pulling on said nose mask during sleep movements of the user;
d) a suspension support bracket and a retractable spring-loaded line mechanism connected to said air hose for suspending said air hose over the user's head while sleeping for relieving said nose mask of any pulling action applied by said suspended air hose during sleep movements of the wearer; and
e) said suspension support bracket being connected to a support base member located under a CPAP machine which acts as a stabilizing counterweight to said suspension support bracket for suspending said air hose over the head of the user.

2. A nose mask assembly in accordance with claim 1, wherein said retractable spring-loaded line mechanism includes a spring-activated pulley having a line thereon.

3. A nose mask assembly in accordance with claim 2, wherein said line in said spring activated pulley is at least 20 inches in length.

4. A nose mask assembly in accordance with claim 3, wherein said line includes a distal end attached to a hose section of said air hose in proximity to said nose mask.

5. A nose mask assembly in accordance with claim 2, wherein said suspension support bracket includes a first end and a second end, wherein said first end of said suspension support bracket is attached to said spring-activated pulley of said retractable spring-loaded line mechanism.

6. A nose mask assembly in accordance with claim 5, wherein said second end of said suspension support bracket is attached to said base plate allowing said air hose to remain suspended above the user.

7. A nose mask assembly in accordance with claim 1, wherein said holding means include an expandable head strap having opposing ends; each of said opposing ends attached to opposing sides of said contoured nose section.

8. A nose mask assembly in accordance with claim 1, wherein said holding means include an head strap having hook and loop straps having opposing proximal ends; each of said opposing proximal ends attached to opposing sides of said contoured nose section.

9. A nose mask assembly in accordance with claim 1, wherein said suspension support bracket is made from materials selected from the group consisting of flexible aluminum tubing, flexible stainless steel tubing, semi-rigid plastic tubing, semi-rigid plastic I-bar and a flat metal bar.

10. A nose mask assembly in accordance with claim 1, wherein said suspension support bracket has a length in the range of 36 inches to 48 inches±3 inches.

11. A nose mask assembly in accordance with claim 1, wherein said suspension support bracket includes a plurality of attachment clamps for detachably attaching said suspension support bracket to said air hose, to said line mechanism and to said support base plate.

12. A nose mask assembly in accordance with claim 1, wherein said support base plate is made from materials selected from the group consisting of metals, plastics or woods being used as a counter-weight to said CPAP machine which sits on said support base plate.

13. A nose mask assembly in accordance with claim 1, wherein said contoured nose section includes a pair of interior nasal openings for allowing air or oxygen to be received through said nasal openings for the wearer.

14. A nose mask assembly in accordance with claim 1, wherein said nose mask is made from moldable thermoplastic materials.

15. A nose mask assembly in accordance with claim 1, wherein said intake port includes connecting means in the form of a threaded opening or a bayonet-type opening.

16. A nose mask assembly in accordance with claim 15, wherein said flexible member includes an insert opening and a threaded male insert element, said threaded male insert element of said flexible member for insertably connecting to said threaded opening of said intake port on said nose mask.

17. A nose mask assembly in accordance with claim 16, wherein said air hose includes a first connecting end and a second connecting end, said first connecting end of said air hose for insertably connecting to said insert opening of said flexible member.

18. A nose mask assembly in accordance with claim 17, wherein said second connecting end of said air hose for insertably connecting to an inlet opening of said CPAP machine for allowing pressurized air to flow from said CPAP machine to said nose mask.

19. A nose mask assembly in accordance with claim 1, wherein said flexible member is a ball joint swivel member for swiveling of said air hose in a side to side motion.

20. A nose mask assembly in accordance with claim 1, wherein said flexible member is a flexible silicone ribbed tubing member for moving of said air hose in a side to side motion.

21. A nose mask assembly in accordance with claim 1, wherein said support base member is a support base plate or a support base bar being located under said CPAP machine.
